SEQUENCE LISTING (10 sequences)

SEQ ID NO: 1 - DNA, 209 bp, human influenza A virus
gagccttctaaccgaggtcgaaacgtatgttctctctatcgttccatcaggccccctcaaagccgaaatcgcgcagagacttgaagatgtctttgctgggaaaaacacagatcttgaggctctcatggaatggctaaagacaagaccaatcctgtcacctctgactaaggggattttggggtttgtgttccctatagtgagtcgtatta

SEQ ID NO: 2 - DNA, 17 bp, Artificial Sequence
  Forward Primer
tcaggccccctcaaagc

SEQ ID NO: 3 - DNA, 21 bp, Artificial Sequence
  Reverse Primer
ccnaaaatccccttagtcaga

SEQ ID NO: 4 - DNA, 21 bp, Artificial Sequence
  Reverse Primer
cccaaaatccccttagtcaga

SEQ ID NO: 5 - DNA, 21 bp, Artificial Sequence
  Reverse Primer
cctaaaatccccttagtcaga

SEQ ID NO: 6 - DNA, 22 bp, Artificial Sequence
  Reverse Primer
caggattggtcttgtctttagc

SEQ ID NO: 7 - DNA, 17 bp, Artificial Sequence
  Detection Probe
cagagacttgaagatgt

SEQ ID NO: 8 - DNA, 14 bp, Artificial Sequence
  Detection Probe
gaggctctcatgga

SEQ ID NO: 9 - DNA, 20 bp, Artificial Sequence
  Consensus sequence for forward primer design
atcaggccccctcaaagccg

SEQ ID NO: 10 - DNA, 50 bp, Artificial Sequence
  Consensus sequence for reverse primer design
tggctaaagacaagaccaatcctgtcacctctgactaaggggattttggg

(19) United States
(12) Patent Application Publication
(10) Pub. No.: US 2010/0055672 A1
(43) Pub. Date: Mar. 4, 2010

(54) 100% SEQUENCE IDENTITY DETECTION METHODS FOR VARIABLE GENOMES

(75) Inventor: Michael G. Saghbini, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: GENEOHM SCIENCES, INC., San Diego, CA (US)

(21) Appl. No.: 12/300,307

(22) PCT Filed: May 10, 2007
(86) PCT No.: PCT/US2007/011401

§ 371 Date: Oct. 15, 2009

Related U.S. Application Data
(60) Provisional application No. 60/799,523, filed on May 11, 2006.

(51) Int. Cl. C12Q 1/70; C07H 21/04; C12P 19/34

(52) U.S. Cl. 435/5; 536/24.33; 435/91.1

(57) ABSTRACT

The present disclosure provides methods, reagents and kits for the detection of all known human variants of influenza A virus and at least 90% of avian and swine variants of influenza A virus in a biological sample, based on amplification primers and detection probes that are specific to a highly conserved region of the influenza A matrix gene.

### CROSS-REFERENCE TO RELATED APPLICATIONS

This application claims priority under 35 U.S.C. §119 to U.S. Provisional Application Ser. No. 60/799,523, filed May 11, 2006 which is incorporated herein by reference in its entirety.

### BACKGROUND OF THE INVENTION

1. Field of the Invention

The present invention relates to detection of influenza A virus variants with 100% sequence identity. More particularly, the invention relates to methods and reagents for detecting influenza A virus in biological samples and to kits for carrying out the methods.

2. Description of the Related Art

Influenza A virus is an RNA virus of the genus Orthomyxovirus, and is the causative agent of influenza, an acute viral infection involving the respiratory tract. It is marked by inflammation of the nasal mucosa, the pharynx, and conjunctiva, and by headache and severe, often generalized, myalgia. Influenza epidemics have been recorded throughout history; the worst of these was the 1918 pandemic, which caused about 20 million deaths worldwide and about 500,000 deaths in the United States. (Sam Baron, MEDICAL MICROBIOLOGY, 4^{th} ed. University of Texas-Galveston (1996)).

Influenza A virus is a single-stranded RNA virus with a segmented genome. The genomic RNAs contain one or more open reading frames flanked by noncoding sequences at the 5′ and 3′ ends (Desselberger et al., Gene 1980, 8:315). Its genetic composition allows this virus to evolve by reassortment of gene segments from different strains; this reassortment creates new variants for which a newly infected organism has no anamnestic immune response. Some influenza A subtypes are species specific, however all subtypes are found in birds (Webster et al., Microbiol. Rev. 1992, 56:152).

Each subtype includes many different strains with new ones arising often due to a highly mutable RNA genome. Of the 15 hemagglutinin (HA) and 9 neuraminidase (NA) subtypes of influenza circulating in aquatic birds, three, H1N1, H2N2, and H3N2 subtypes are known to have caused pandemics in humans (Webster et al., Microbiol. Rev. 1992, 56:152). There is also evidence that pigs can serve as an intermediate host (“mixing vessel”) for the generation of new strains that are pathogenic in humans (Scholtissek et al., Virology 1985, 147:287). The avian virus H5N1 , which caused the deadly “avian flu” outbreak in Hong Kong in 1997, showed that highly pathogenic influenza A viruses can also be transmitted directly from avian species to humans (Claas et al., Lancet 1998, 351:472; Suarez et al., J. Virol. 1998, 72:6678; Subbarao et al., Science 1998, 279:393; Shortridge, Vaccine 1999, 17 (Suppl. 1): S26; Webby and Webster, Science 2003, 302:1519).

Treatment for influenza A is available through antiviral therapies such as neuraminidase inhibitors. These antiviral treatments are most effective when administered within the first 48 hours after the onset of illness. Hayden et al., J. Am. Med. Assoc. 1999; 282:1240. Yet traditional methods of detecting the presence of influenza A virus strains involve several lengthy steps, including propagation in cell lines or in embryonated eggs. Viral antigens are then detected in the cell cultures using fluorescent- or immunoperoxidase-labeled antibodies specific for influenza virus. A major drawback of these traditional culture methods is that they often take several days and do not provide results in a time frame that is clinically relevant, i.e. within the first 48 hours after the onset of symptoms. See Newton et al., Am. J. Manag. Care 2000, 6:S265.

Further efforts to decrease the turnaround time of laboratory diagnosis have utilized molecular methods to detect viral nucleic acids in patient specimens. Nucleic acid amplification methods provide a better sensitivity than traditional virus culture and immunofluorescence techniques. See Ellis and Zombon, Rev. Med. Virol., 2002, 12:375. Amplification-based methods typically involve two basic steps: 1) amplification with a set of oligonucleotide primers to generate a specific amplicon, and 2) detection of the amplicon, preferably employing sequence specific hybridization probes, to signal the presence of the virus.

Because the many different strains of influenza A exhibit multiple nucleotide differences at the genome level, existing methods have placed the amplification primers and detection probes in the most conserved nucleic acid regions. Fouchier et al., J. Clin. Microbiol. 2000, 38:4096; Spackman et al., Avian Dis. 2003, 47:1079. However, it is impossible to identify such regions having 100% sequence homology across the already existing human influenza A variants (>450 sequences), let alone the many different other species variants, especially birds (>650 avian variants), which can cause severe disease in humans. The use of degenerate oligonucleotides to achieve 100% sequence homology to existing virus variants greatly increases the oligonucleotide complexity since variability occurs in different parts of a given region for different variants. Suarez and Perdue, Virus Res. 1998, 54:59; Xu et al., Virology 1996, 224:175. This leads to the use of multiple detection probes and a reduction in detection sensitivity.

As such, current amplification-based detection methods for the detection of human influenza A variants lack the ability to detect all existing human variants and a number of avian variants based on 100% sequence homology for amplification and detection probes, a desirable process for diagnostic product development. Instead, the extent of detection in existing methods is a function of the degree of sequence homology of the amplification and detection oligonucleotides to the virus variant.

Accordingly, there is an unmet need in the art for nucleic acid detection methods that achieve 100% theoretical detection of all influenza A variants.

### SUMMARY OF THE INVENTION

Detection methods for variable genomes and reagents for the same are disclosed. The invention allows for low cost, reliable detection of the presence of at least 90% and preferably 100% of human variants of influenza A virus and at least 80% and more preferably at least 90% of avian and swine variants of influenza A virus in a biological sample, based on amplification primers that are specific to a highly conserved region of the influenza A matrix gene.

In accordance with the above, provided herein is a set of polynucleotides that includes at least one forward primer and at least one reverse primer that are each substantially the same as or substantially complementary to a portion of the target nucleic acid having the sequence shown in SEQ ID NO: 1, such that the set of polynucleotides is capable of amplfiying at least 90% of human, swine and avian variants of influenza A virus. In certain aspects, the variants of influenza A are selected from a group comprising: human influenza A, avian influenza A or swine influenza A. In yet another aspect, the variants of influenza A comprise any combination of human influenza A, avian influenza A and swine influenza A. In a further embodiment, each primer is at least 10, at least 11, at least 12, at least 13, or at least 15 nucleotides in length. In certain aspects, each primer is between 15 and 30 nucleotides in length.

In certain embodiments, the forward primer comprises the nucleic acid molecule of SEQ ID NO:2 and the reverse primer is the nucleic acid molecule selected from the group comprising SEQ ID NOs:3, 4, 5 or 6. In another embodiment, the forward primer comprises at least 10 consecutive nucleotides of SEQ ID NO:2 and the reverse primer comprises at least 10 consecutive nucleotides selected from the group comprising SEQ ID NOs:3, 4, 5 or 6.

Further provided herein is a kit for amplification of influenza A virus variants which includes a pair of primers that have nucleotide sequences substantially complementary to the influenza A viral nucleic acid or the complement of the nucleic acid, which are adapted to participate in the generation of an amplification product from a target nucleic acid. In a preferred embodiment, this pair of primers is capable of amplifying at least 90% of human variants of influenza A virus and at least 80% of avian and swine variants of influenza A virus. Preferably, at least one pair of primers comprises one of the following pairs:
i) SEQ ID NO:2 and SEQ ID NO:3,
ii) SEQ ID NO:2 and SEQ ID NO:4,
iii) SEQ ID NO:2 and SEQ ID NO:5, or
iv) SEQ ID NO:2 and SEQ ID NO:6.

In a preferred embodiment, the kit further comprises a nucleic acid consisting of a portion of the sequence shown in SEQ ID NO:1, and further comprises a panel of at least two detection probes wherein at least one of the detection probes is complementary to at least a portion of a sequence in the amplification product.

In certain aspects, the panel comprises at least one detection probe that is complementary to the sequence of at least one influenza A target to be amplified for each virus of said variants of influenza A. In a further embodiment, the panel comprises at least one detection probe that is substantially complementary to the sequence of at least one influenza A target to be amplified for each virus of said variants of influenza A.

Another aspect of the invention is a method of detecting the presence of at least 90% of variants of influenza A virus in a biological sample. In a preferred embodiment, target nucleic acid from the biological sample is then amplified using primer pairs directed against a region of the viral genome that is conserved across more than at least two variants of the virus, such that at least one forward primer and at least one reverse primer in combination will bind to the target genome of said variants. Amplified target nucleic acid is then detected by placing any amplified nucleic acid in contact with a panel of detection probes wherein at least one of the detection probes is complementary to at least a portion of the sequence in the amplified nucleic acid, and then determining whether a signal indicative of the presence of said target nucleic acid in said sample has been generated.

Another aspect of the invention is a method of detecting the presence of at least 90% of variants of influenza A virus in a biological sample comprising: amplifying at least one influenza A target nucleic acid using at least one primer directed against a conserved region of the viral genome, wherein said region is conserved across more than at least two variants of the virus; and detecting any amplified target nucleic acid. In a preferred embodiment, said variants of influenza A are selected from a group comprising: human influenza A, avian influenza A or swine influenza A. Preferably, said at least one primer comprises: SEQ ID NO:2; SEQ ID NO:3; SEQ ID NO:4; SEQ ID NO:5; or SEQ ID NO: 6.

In some aspects of the above embodiments, amplified target nucleic acid is detected by: placing any amplified nucleic acid in contact with a panel of detection probes wherein at least one of the detection probes is complementary to at least a portion of a sequence in the amplified nucleic acid; and determining whether a signal indicative of the presence of said target nucleic acid in said sample has been generated.

In another aspect, the panel comprises at least one detection probe that is complementary to the sequence of at least one influenza A target to be amplified, for each virus of said variants of influenza A.

In certain aspects, the panel comprises at least one detection probe that is substantially complementary to the sequence of at least one influenza A target to be amplified, for at least one virus of said variants of influenza A.

In certain aspects, at least one of said detection probes is complementary to at least a portion of a nucleic acid having the sequence or the complement of said conserved region.

In one embodiment, at least one of these detection probes is complementary to the conserved region of influenza A virus matrix protein. In another embodiment, at least one of these detection probes is complementary to one or more tag sequences in the amplified DNA. In another embodiment, the detection probes themselves comprise both sequence complementary to the amplified target DNA and a tag sequence capable of hybridizing to a universal detector probe.

In a further embodiment, at least one of said detection probes comprises at least 10 consecutive nucleotides selected from the group comprising SEQ ID NOs: 7 or 8.

In some embodiments, the signal indicative of the presence of the target nucleic acid is generated by a catalytic detection reagent which can produce a plurality of signals without being exhausted. In a preferred embodiment, the conserved region of the target nucleic acid comprises a portion of the sequence shown in SEQ ID NO: 1. Preferably, at least one pair of primers comprises one of the following pairs:
i) SEQ ID NO:2 and SEQ ID NO:3,
ii) SEQ ID NO:2 and SEQ ID NO:4,
iii) SEQ ID NO:2 and SEQ ID NO:5, or
iv) SEQ ID NO:2 and SEQ ID NO:6.Another aspect is a kit for detecting influenza A virus, comprising at least one primer capable of participating in the production of an amplification product; and a detection reagent capable of detecting the formation of the amplification product.

Another aspect is a kit for detecting influenza A virus, comprising a set of primers capable of participating in the production of an amplification product; and a detection reagent capable of detecting the formation of the amplification product.

### BRIEF DESCRIPTION OF THE DRAWINGS

FIG. 1 illustrates the results of comparative sequence analysis of over 1000 variants of influenza A virus, and shows the 100% conserved region of the matrix protein of influenza A virus. FIG. 1A shows the analysis for the forward primer region, shown in the sequence listing as SEQ ID NO: 9. FIG. 1B shows the analysis for the reverse primer region, shown in the SEQ ID NO: 10. The first column denotes the host species (swine, avian) or human subtype (e.g. H5N1), and the second column indicates the number of variants within that category that were analyzed. The bottom row indicates the consensus sequence, and locations of primer design are shaded.

FIG. 2 illustrates one mode of the invention. Forward primers, reverse primers, and detection oligonucleotides are indicated.

FIG. 3 illustrates the results of one experiment according to the invention. Shown is a graph of electrochemical detection signal in relative units for each of a series of different detection probes.

### DETAILED DESCRIPTION OF THE PREFERRED EMBODIMENT

### Advantages

The present disclosure is generally related to methods for detecting influenza A variants in biological samples. Advantages over the prior art include detection of all known human influenza A variants with a high probability of detecting new variants. Additionally advantageous is the detection of more than 90% of all avian and swine variants, including all avian H5N1 variants.

The present disclosure also has the advantage of higher detection sensitivity of influenza A virus variants. The prior art utilizes complex degenerate primers and multiple detection probes in order to amplify and detect all known variants, and the result is an increase in noise and a reduction in sensitivity. However, the present disclosure achieves higher detection sensitivity by means of lower complexity of both amplification oligonucleotides and detection probes.

Finally, the universal nature of the present disclosure is better suited for development of related detection technologies in terms of scope of detection and sensitivity validation studies. The prior art has the disadvantage of requiring that all known variants with differences from consensus sequences be tested.

### Definitions and Abbreviations

“Amplification reagents” designates collectively the various buffers, enzymes, primers, deoxynucleoside triphosphates, and oligonucleotides used to perform the selected PCR or RT-PCR amplification procedure.

“Amplifying” or “Amplification” means any suitable method of amplifying a nucleic acid that employs a specific polynucleotide probe or primer. Suitable techniques known in the art include Polymerase Chain Reaction (PCR), Transcription Mediated Amplification (TMA), Oligonucleotide Ligation Assay (OLA), Ligase Chain Reaction (LCR), Rolling Circle Amplification (RCA) and others. However, these terms preferably refer to any essentially quantitative and preferably logarithmic increase in a target sequence as a result of a PCR designed to amplify the specific target sequence. “Amplicon” means an amplification product.

“Anneal” refers to complementary hybridization between an oligonucleotide and a target sequence and embraces minor mismatches that can be accommodated by reducing the stringency of the hybridization to achieve the desired priming for the reverse transcriptase or DNA polymerase or for detecting a hybridization signal.

“Biological sample” designates anything suspected of containing a target sequence. The biological sample can be derived from any biological source without limitation. A biological sample can be used (i) directly as obtained from the source; or (ii) following a pre-treatment to modify the character of the test sample. Thus, the biological sample can be pre-treated prior to use by, for example, disrupting cells and/or virions, preparing liquids from solid biological samples, diluting viscous fluids, filtering liquids, distilling liquids, concentrating liquids, inactivating interfering components, adding reagents, purifying nucleic acids, and the like.

“cDNA” refers to complementary or copy DNA produced from an RNA template by the action of RNA-dependent DNA polymerase (reverse transcriptase).

“Complementary” refers to polynucleotides that are capable of hybridizing, e.g. sense and anti-sense strands of DNA or self-complementary strands of RNA, due to complementarity of aligned nucleotides permitting C-G and A-T or A-U bonding.

“Consensus sequence” refers to a way of representing the results of a multiple sequence alignment, where related sequences are compared to each other, and similar functional sequence motifs are found. The consensus sequence shows which residues are conserved (are always the same), and which residues are variable.

“Detection probe” generally refers to a molecule capable of binding to a target sequence or a tag, where “detection probe” may encompass probe molecules immobilized to a support and probe molecules not immobilized to a support. More specifically, the term “probe sequence” as used herein refers to the nucleotide sequence of an oligonucleotide probe, where “probe sequence” may describe a physical string of nucleotides that make up a sequence, or may describe an information string representing the properties of the string of nucleotides, where such an information string can be manipulated as part of a program for designing or selecting a set of probes having desired properties. The term “detection probe” is generally used herein to refer to a tag-complementary probe coupled to a detection means for measuring hybridization of a tag to the detection probe.

“Hybridization” refers to the formation of a duplex structure by two single stranded nucleic acids due to complementary base pairing. Hybridization can occur between exactly complementary nucleic acid strands or between nucleic acid strands that contain minor regions of mismatch. As used herein, the term “substantially complementary” refers to sequences that are complementary except for minor regions of mismatch, wherein the total number of mismatched nucleotides is no more than about 3 for a sequence about 15 to about 35 nucleotides in length. Conditions under which only exactly complementary nucleic acid strands will hybridize are referred to as “stringent” or “sequence-specific” hybridization conditions. Stable duplexes of substantially complementary nucleic acids can be achieved under less stringent hybridization conditions. Those skilled in the art of nucleic acid technology can determine duplex stability empirically considering a number of variables including, for example, the length and base pair concentration of the oligonucleotides, ionic strength, and incidence of mismatched base pairs. Computer software for calculating duplex stability is commercially available from a variety of vendors.

“Nucleic acid” refers to a deoxyribonucleotide or ribonucleotide polymer in either single- or double-stranded form, and unless otherwise limited, encompasses known analogs of natural nucleotides that can function in a manner identical to or similarly to naturally occurring nucleotides.

“Oligonucleotide” or “Polynucleotide” refers to a molecule comprised of two or more deoxyribonucleotides or ribonucleotides, such as primers, probes, nucleic acid fragments to be detected, and nucleic acid controls. The exact size of a polynucleotide depends on many factors and the ultimate function or use of the polynucleotide. Polynucleotides can be prepared by any suitable method now known in the art or developed in the future, including, for example, using conventional and well-known nucleotide phosphoramidite chemistry and the instruments available from Applied Biosystems, Inc, (Foster City, Calif.); Dupont, (Wilmington, Del.); or Milligen, (Bedford, Mass).

“PCR” designates the polymerase chain reaction.

“RT-PCR” designates the reverse-transcriptase-polymerase chain reaction.

“Reverse transcriptase” refers to an enzyme that catalyzes the polymerization of deoxyribonucleoside triphosphates to form primer extension products that are complementary to a ribonucleic acid template. The enzyme initiates synthesis at the 3′-end of the primer that is annealed to the RNA template and proceeds toward the 5′-end of the RNA template until synthesis terminates.

“Primer” refers to an oligonucleotide, whether natural or synthetic, capable of acting as a point of initiation of DNA synthesis under conditions in which synthesis of a primer extension product complementary to a nucleic acid strand is induced, i.e., in the presence of four different nucleoside triphosphates and an agent for polymerization (i.e., DNA polymerase or reverse transcriptase) in an appropriate buffer and at a suitable temperature. A primer is preferable a single-stranded oligodeoxyribonucleotide. The appropriate length of a primer depends on the intended use of the primer but typically ranges from 15 to 30 nucleotides and can be as short as 8 nucleotides and as long as 50 or 100 nucleotides. Short primer molecules generally require cooler temperatures to form sufficiently stable hybrid complexes with the template. A primer need not reflect the exact sequence of the template but must be sufficiently complementary to hybridize with the template. Primers may be “forward” or “reverse.” A forward primer refers to the primer used to initiate synthesis of the strand in which the primer is incorporated. A reverse primer refers to the primer used to initiate synthesis of the strand which is complementary to the strand whose synthesis was initiated by the forward primer.

“Strain” and “subtype” refers to classification of influenza type A viruses. Currently, there are 15 subtypes of type A influenza, classified by the hemagglutinin (HA) and neuraminidase (NA) surface glycoproteins. Within each subtype, there are many strains or variants that have variations at the nucleotide level.

“Tag” generally refers to a molecule capable of binding to a probe, where “tag” may encompass tag molecules attached to a target molecule, tag molecules not attached to target molecules, tags expressed in computer-readable form, and the concept of tags as disclosed herein. The term “tag sequence” as used herein refers to the nucleotide sequence of an oligonucleotide tag, where “tag sequence” or “identifier tag sequence” may describe a string of nucleotides or may describe an information string representing the properties of the string of nucleotides, where such an information string can be manipulated as part of a program for designing or selecting a set of tags having desired properties. In the present invention, an “identifier tag” is a tag chosen to serve as a distinct identifier for a particular target. As used herein, the term “identifier tag” is used to refer both to the oligonucleotide that binds to a complementary detection probe and to nucleotide sequence of the identifier tag. The term “complement of an identifier tag” can refer to a string of nucleotides that make up the oligonucleotide having a nucleotide sequence complementary to the nucleotide sequence of the identifier tag, and can also refer to the nucleotide sequence (information string) of the complement.

“Target sequence” or “target region” are synonymous terms and designate a nucleic acid sequence (single- or double-stranded) that is detected and/or amplified, or will otherwise anneal under stringent conditions to one of the primers or probes herein provided.

“Thermostable polymerase” refers to an enzyme that is relatively stable to heat and catalyzes the polymerization of nucleoside triphosphates to form primer extension products that are complementary to one of the nucleic acid strands of a target sequence. The enzyme initiates synthesis at the 3′-end of the primer that is annealed to the template and proceeds toward the 5′-end of the template until synthesis terminates. A purified thermostable polymerase enzyme is described more fully in U.S. Pat. Nos. 4,889,818 and 5,079,352. The term encompasses polymerases that have reverse transcriptase activity. Numerous thermostable polymerases are available from a host of commercial suppliers, such as Applied Biosystems and Promega Corporation, Madison, Wis.

“Transcript” refers to a product of RNA polymerase, typically a DNA dependant RNA polymerase.

### Description

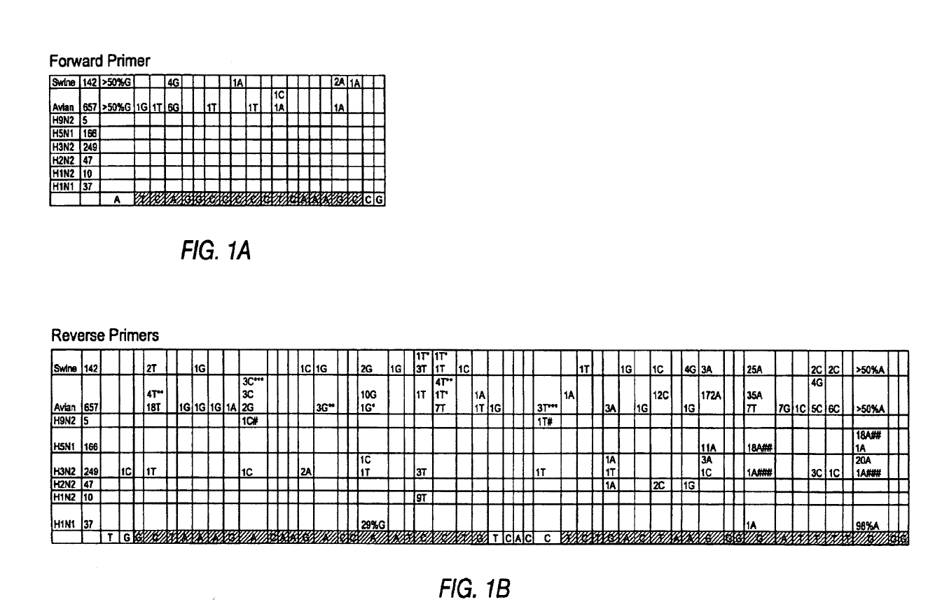

One aspect provides polynucleotides capable of detecting at least 90%, and preferably 100% of human variants of influenza A virus; and at least 80% but preferably 90% of avian and swine variants of influenza A virus. Accordingly, the polynucleotides are directed to a highly-conserved region of the influenza A virus matrix protein gene (FIG. 1). Careful nucleic acid sequence analysis of the matrix protein gene from the over 1000 influenza A variants identified from human and other animals, revealed that differences among variants within the matrix protein gene are limited. As such, it is possible to identify conserved regions that have within their boundaries differences for the various variants in different positions but between them achieve 100% sequence homology to at least all human variants. Thus, for any given virus variant, there is always at least one region that shares 100% homology to the other variants. Placing the amplification and detection probes in 100% homologous regions assures 100% theoretical detection of all variants.

### Identification of Primer and Detection Probe Sequences

FIG. 1 illustrates the results of comparative sequence analysis of over 1000 variants of influenza A virus. In both FIGS. 1A and 1B, each row denotes nucleotide differences identified in swine, avian, and human variants of the virus, compared to the consensus sequence (bottom row). For example, “4G” (FIG. 1A, top row) indicates that four of the 142 swine variants contain a guanosine (G) nucleotide at this position instead of an adenosine (A) nucleotide. Some variants have more than one base substitution and are denoted by “*” and “#” symbols (FIG. 1B).

In a preferred embodiment, the forward primer is complementary to a region of the influenza A matrix protein that contains no known nucleotide differences among all the human variants (FIG. 1A, shaded sequence). The forward primer is also capable of detecting over 90% of avian and swine variants, as demonstrated by the small number of nucleotide differences in this region of some avian and swine variants (FIG. 1A).

One aspect of the invention utilizes reverse primers that are capable of detecting all known human variants, and over 90% of avian and swine variants (FIG. 1B). In a preferred embodiment, one reverse primer with a three-base “wobble” near the 5′ end is capable of detecting over 90% of all known human, avian and swine variants (SEQ ID NOs:3-5). In the sequence of SEQ ID NO: 3, N stands for inosine. A second reverse primer (SEQ ID NO. 6) is by itself capable of detecting over 90% of all know human, avian and swine variants.

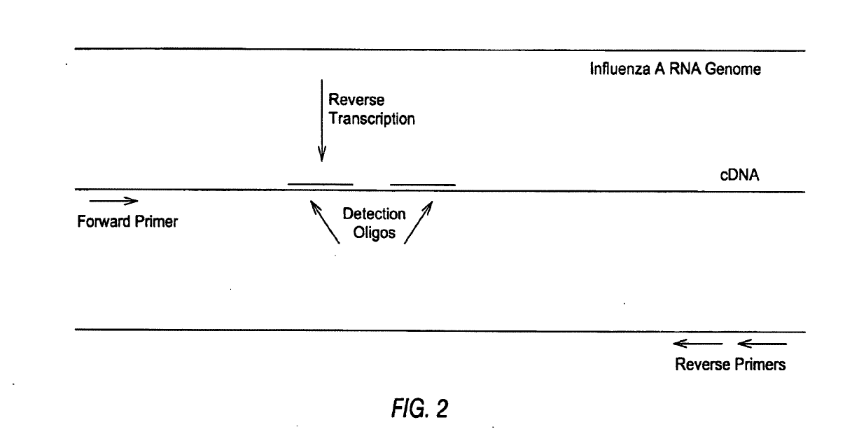

In a preferred embodiment (FIG. 2), one forward primer is used in combination with reverse primers designed to bind to at least two different regions in order to achieve 100% theoretical amplification of all human variants. Additionally, as shown in FIG. 2, at least two regions have been identified for the detection probes (SEQ ID NOs:7 and 8), such that 100% theoretical detection of all human variants is achieved. In this embodiment, over 90% of avian variants and over 90% of swine variants are amplified and detected.

In some embodiments, at least two regions for the forward and reverse primers and at least two regions for the detection probes are used, whereby at least two separate amplicons are generated with at least one detection probe capable of detecting each amplicon.

In other embodiments, at least one region is used for the reverse primer in combination with at least two regions for the forward primers and at least two regions for the detection probes.

In other embodiments, at least one region is used for the detection probe in combination with at least two regions for the forward primers and at least two regions for the reverse primers.

In other embodiments, at least one region is used for the forward primers and the reverse primers in combination with at least two regions for the detection probes.

In other embodiments, at least one region is used for the forward primer and detection probe in combination with at least two regions for the reverse primers.

In other embodiments, at least one region is used for the detection probe and the reverse primer in combination with at least two regions for the forward primers.

### Target Sequences

In some embodiments, the target nucleic acid is a synthetic oligonucleotide that serves as a template for primer binding. A synthetic oligonucleotide template may serve as a control for amplification, and alleviates the need for acquiring outside viral RNA sources for initial development or optimization of amplification conditions. Additionally, such a synthetic oligonucleotide template facilitates a scan of candidate detection probes without requiring RT-PCR generation of template from viral RNA.

In some embodiments, the synthetic oligonucleotide is a single stranded oligonucleotide that spans the entire target region. In other embodiments, the synthetic template is double stranded, generated from two overlapping complementary strands where the ends are made double stranded using a DNA polymerase in an end-fill reaction, as is known to those skilled in the art. In preferred embodiments, the synthetic target nucleic acid contains a T7 promoter in the 3′ end to facilitate in vitro generation of RNA templates for RT-PCR.

In some embodiments, the target nucleic acid contains sequence that is substantially the same as a specific variant of influenza A virus. In other embodiments, the target nucleic acid consists of the entire influenza A RNA genome from a specific variant. In still other embodiments, the target nucleic acid is a synthetic oligonucleotide that contains the consensus sequence from a region conserved among either a set of influenza A variants, a group of strains, or among all known strains. In a preferred embodiment, the target nucleic acid contains sequence that is the same as or substantially the same as SEQ ID NO: 1.

The target nucleic acid can be cDNA generated from viral RNA collected from a biological sample. Methods, techniques and reagents for isolating RNA from biological samples and for converting viral RNA to cDNA are well known to those of skill in the art. (See e.g., Sambrook et al. MOLECULAR CLONING: A LABORATORY MANUAL, 3^{rd} ed., Cold Spring Harbor Laboratory Press, Cold Spring Harbor, N.Y., (2001) or Ausubel et al., CURRENT PROTOCOLS IN MOLECULAR BIOLOGY, J. Wiley and Sons, New York, N.Y. (1992))

### Amplification of Target Sequences

One aspect of the invention involves amplification of target sequences using well-known methods to generate amplification products that include only the target sequence. Optionally, amplification products contain additional exogenous nucleotide sequences, such as tag sequences, involved in post-amplification manipulation of the amplification product without a significant effect on the amplification step itself. Linear or exponential (nonlinear) modes of amplification may be used with any suitable amplification method, where choice of mode is made by one of skill in the art depending on the circumstances of a particular embodiment. Methods of amplification include, but are not limited to, use of polymerase chain reaction (PCR) and rolling circle (RC) amplification to amplify polynucleotide templates.

Template amplification by polymerase chain reaction (PCR) uses multiple rounds of primer extension reactions in which complementary strands of a defined region of a DNA molecule are simultaneously synthesized by a thermostable DNA polymerase. During repeated rounds of primer extension reactions, the number of newly synthesized DNA strands increases exponentially such that after 20 to 30 reaction cycles, the initial template can be replicated several thousand-fold or million-fold. Methods for carrying out different types and modes of PCR are thoroughly described in the literature, for example in “PCR Primer: A Laboratory Manual” Dieffenbach and Dveksler, Eds. Cold Spring Harbor Laboratory Press, 1995, and by Mullis et al. in patents (e.g., U.S. Pat. Nos. 4,683,195, 4,683,202 and 4,800,159) and scientific publications (e.g. Mullis et al. 1987, Methods in Enzymology, 155:335-350), and in U.S. Pat. No. 6,815,167.

Briefly, PCR proceeds in a series of steps as described below. In the initial step of the procedure, double-stranded template is isolated and heat, preferably between about 90° C. to about 95° C., is used to separate the double-stranded DNA into single strands (denaturation step). The initial denaturation step is omitted for single-stranded template. Cooling to about 55° C. allows primers to adhere to the target region of the template, where the primers are designed to bind to regions that flank the target nucleic acid sequence (annealing step). Thermostable DNA polymerase (e.g., Taq polymerase) and free nucleotides are added to create new DNA fragments complementary to the target region of the template via primer extension (extension step), to complete one cycle of PCR. This process of denaturation, annealing and extension is repeated numerous times, preferably in a thermal cycler. At the end of each cycle, each newly synthesized DNA molecule acts as a template for the next cycle, resulting in the accumulation of many hundreds or thousands, or even millions, of double-stranded amplification products from each template molecule.

In multiplex PCR, the assay is modified to include multiple primer pairs specific for distinct target nucleotide sequences of the same template, to allow simultaneous amplification of multiple distinct target nucleotide sequences and generation of multiple distinct single-stranded DNA molecules having the desired nucleotide sequence and length. For example, multiplex PCR can be carried out using the genomic DNA of an organism or an individual as the template, where multiplex PCR will produce multiple distinct single-stranded DNA molecules.

PCR generates double-stranded amplification products suitable for post-amplification processing. PCR amplification products may contain features such as additional nucleotide sequences not found in the target nucleotide sequence. Primers used to amplify template may be designed to introduce features into amplification products by introducing exogenous nucleotide sequence(s) not found in the target nucleotide sequence. Such features include, but are not limited to, identifier tags, restriction digestion sites, modified nucleotides, promoter sequences, inverted repeats, chemical modifications, addressable ligands, and other non-template 5′ extensions that allow post amplification manipulation of amplification products without a significant effect on the amplification itself. Preferably, the exogenous sequences are 5′ (“upstream”) of the primer sequence involved in binding to the target nucleotide sequence. In one preferred embodiment, primers introduce identifier tags. In another embodiment, primers introduce sites involved in restriction enzyme recognition, binding and cleavage (“trimming”) of amplification products.

In another aspect, amplification methods other than PCR are used to amplify the target sequence. Alternative methods of amplification include, but are not limited to, ligase chain reaction (LCR) and rolling circle amplification (RCA). Protocols for carrying out these and other amplification methods are well known in the art, particularly as described by Xu and Kool (1999, Nuc Acids Res 27:875-881), Kool et al. (U.S. Pat. Nos. 5,714,320, 6,368,802 and 6,096,880), Landegren et al. (U.S. Pat. No. 5,871,921), Zhang et al. (U.S. Pat. Nos. 5,876,924 and 5,942,391) and Lizardi et al. (Lizardi et al., 1998, Nature Genet 19: 225-232, and U.S. Pat. Nos. 5,854,033, 6,124,120, 6,143,495, 6,183,960, 6,210,884, 6,280,949, 6,287,824, and 6,344,329).

In another aspect, alternative methods of amplification may include use of single primer amplification (SPA) or use of scorpion primer amplification and detection. U.S. Pat. No. 6,326,145, hereby incorporated by reference in its entirety, describes a method for the detection of a target nucleic acid, including contacting template nucleic acid with a tailed nucleic acid primer having a template binding region and the tail comprising a linker and a target binding region. During amplification, the template binding region of the primer hybridizes to a complementary sequence in the template nucleic acid and is extended to form a primer extension product, separating any such product from the template whereupon the target binding region in the tail of the primer hybridizes to a sequence in the primer extension product corresponding to the target nucleic acid. The target nucleic acid in the sample is detected by a change in the signal of a signaling system, for example an attached fluorophore and a proximal quencher molecule.

### Detection of Amplification Products

One aspect of the invention involves detection of amplification products using detection oligonucleotides or “probes”. As discussed herein, regions of the target sequence have been identified that allow for 100% theoretical amplification and detection of all human-borne strains of influenza A and at least 90% of avian and swine strains. Detection probes may contain sequence identical to or substantially the same as the target sequence. Alternatively, detection probes may contain sequence that is complementary or substantially complementary to the target sequence. In some embodiments, more than one region is used for design of detection probes in order to obtain 100% theoretical detection of amplified target sequences (See FIG. 2).

In some embodiments, detection probes are placed in contact with a sample containing the amplified target sequence under conditions which permit the amplified target sequence to hybridize to the detection probe. Hybridization of the target sequence to the detection probe is then measured. For example, the detection probes may be directly immobilized on an assay chip that contains an electrode surface capable of signaling when hybridization takes place. Alternatively, the detection probes may include tag sequences which are further amplified through RCA and which then hybridize to an immobilized probe on a universal assay chip. In these examples, hybridization to an immobilized probe strand can be detected using several different techniques.

Various techniques and electron transfer species useful for nucleic acid detection are disclosed in WO 2004/044549; U.S. patent application Ser. No. 10/424,542 entitled “UNIVERSAL TAG ASSAY,” filed Apr. 24, 2003. Specifically, one aspect discussed in these applications is detection of hybridization of tags and immobilized probes using a transition metal complex capable of oxidizing at least one oxidizable base in an oxidation-reduction reaction.

Further embodiments are discussed in U.S. patent application Ser. No. 10/429,291, entitled “ELECTROCHEMICAL METHOD TO MEASURE DNA ATTACHMENT TO AN ELECTRODE SURFACE IN THE PRESENCE OF MOLECULAR OXYGEN,” filed May 2, 2003; U.S. patent application Ser. No. 10/429,293, entitled “METHOD OF ELECTROCHEMICAL DETECTION OF SOMATIC CELL MUTATIONS,” filed May 2, 2003; and co-pending PCT Application No. PCT/US2004/027412, filed Aug. 23, 2004.

Specifically, U.S. patent application Ser. No. 10/429,293 discusses methods of enhancing the signal by elongating the target strand after it has hybridized to the probe strand, a technique sometimes referred to as “on-chip amplification.”

One example of an assay that utilizes universal tag detection is disclosed in U.S. patent application Ser. No. 10/985,256 entitled “NUCLEIC ACID DETECTION METHOD HAVING INCREASED SENSITIVITY.” This application discusses nucleic acid detection methods having increased sensitivity by utilizing electrochemical detection of a catalytic cycle between a synthetically elongated nucleic acid and an electrode surface. Specifically, this application discusses embodiments that include the use of catalytic detection moieties instead of counterions (such as ruthenium complexes) which themselves undergo electron transfer at an electrode surface.

### Examples

The following example is included for illustrative purposes only and is not intended to limit the scope of the invention.

### Example 1

A ˜200 bp synthetic RNA corresponding to a portion of the influenza A matrix gene of interest (SEQ ID NO: 1) was generated by designing overlapping oligos and filling the ends with a 2 hours Taq polymerase extension at 37° C. The resulting double stranded template had a T7 RNA polymerase promoter on one end enabling the production of the synthetic RNA following a standard transcription reaction. The RNA template was quantitated on an Agilent 2100 Bioanalyzer to estimate an RNA copy number.

Reverse transcription PCR was carried out on 300-500 RNA copies, digested with RNAse-free DNAse I to ensure removal of ds DNA template, using Qiagen One-Step RT-PCR kit, according to manufacturer's instructions. Gel results confirmed transcription. The rt-PCR mix consisted of 0.2-0.4uM 5′ phosphorylated forward primer (SEQ ID NO: 2) and 5′ FAM labeled reverse primers (SEQ ID NOS: 5 and 6), 2 uM MgCl2, 1x rt-PCR mix, 0.1 mM dNTP, 10 units RNasin (Promega) and 5 units Qiagen enzyme mix, in a final volume of 50 ul. Amplification conditions were as follow: 50° C. for 30 min, 95° C. for 15 min, 40 cycles of [95° C. for 30 sec, 55° C. for 30 sec, 72° C. for 1 min], and a final incubation at 72° C. for 10 min with storage at 4° C.

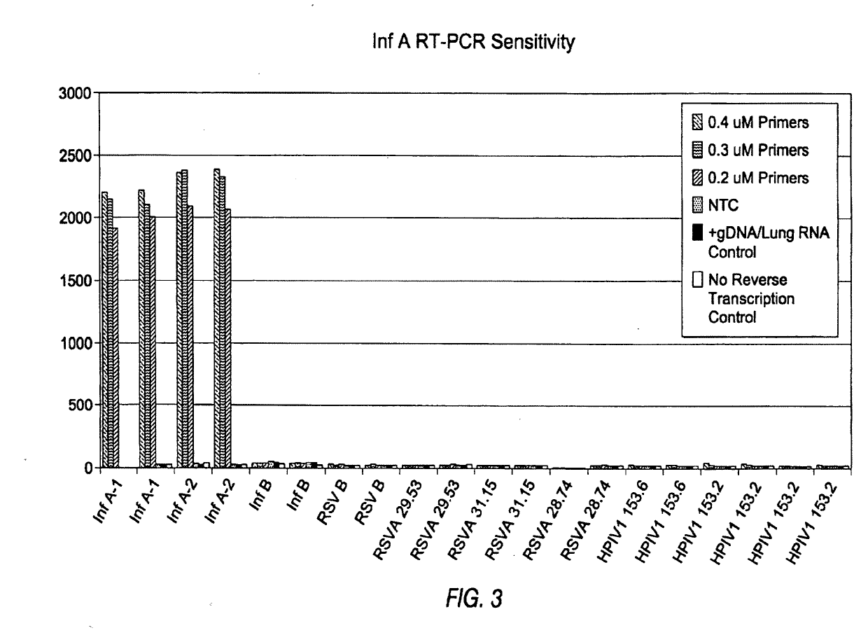

The PCR product was then digested with λ exo to produce a single stranded template. The template was subsequently detected using an ePlex™ electrochemical detection platform (GeneOhm Sciences, San Diego, Calif.) using Influenza A detection probes (SEQ ID NO: 7 and 8). Results are shown in FIG. 3. Compared to negative controls, the amplification produced a product that was specifically detected by Influenza-A detection probes.

The above description of the disclosed embodiments is provided to enable any person skilled in the art to make or use the invention. Various modifications to these embodiments will be readily apparent to those skilled in the art, and the generic principles defined herein may be applied to other embodiments without departing from the spirit or scope of the invention. Thus, the invention is not intended to be limited to the embodiments shown herein but is to be accorded the widest scope consistent with the principles and novel features disclosed herein.

### INCORPORATION BY REFERENCE

All references cited herein, including patents, patent applications, papers, text books, and the like, and the references cited therein, to the extent that they are not already, are hereby incorporated herein by reference in their entirety.

What is claimed is:

1. A set of polynucleotides, comprising:
at least one forward primer that is substantially the same as or substantially complementary to a portion of the target nucleic acid having the sequence shown in SEQ ID NO: 1; and
at least one reverse primer that is substantially the same as or complementary to said target nucleic acid; and
wherein said set of polynucleotides is capable of amplifying at least 90% of human, swine and avian variants of influenza A virus.

2. The set of polynucleotides of claim 1, wherein said variants of influenza A are selected from a group comprising: human influenza A, avian influenza A or swine influenza A.

3. The set of polynucleotides of claim 1, wherein said variants of influenza A comprises any combination of human influenza A, avian influenza A and swine influenza A.

4. The set of polynucleotides of claim 1, wherein the forward and reverse primers each comprise at least 10 consecutive nucleotides.

5. The set of polynucleotides of claim 1, wherein each primer is 15 to 30 nucleotides in length.

6. The set of polynucleotides of claim 2, wherein the forward primer comprises the nucleic acid molecule of SEQ ID NO:2 and the reverse primer is the nucleic acid molecule selected from the group comprising SEQ ID NOs:3, 4, 5 or 6.

7. The set of polynucleotides of claim 2, wherein the forward primer comprises at least 10 consecutive nucleotides of SEQ ID NO:2 and the reverse primer comprises at least 10 consecutive nucleotides selected from the group comprising SEQ ID NOs:3, 4, 5 or 6.

8. A kit for amplification of influenza A virus variants, the kit comprising:
a pair of primers that have nucleotide sequences substantially complementary to the nucleic acid or the complement of the nucleic acid, said primers adapted to participate in the generation of an amplification product from a target nucleic acid,
wherein said pair of primers is capable of amplifying at least 90% of human variants of influenza A virus; and
wherein said pair of primers is capable of amplifying at least 80% of avian and swine variants of influenza A virus.

9. The kit as claimed in claim 8, wherein at least one pair of primers comprises:
a) SEQ ID NO:2 and SEQ ID NO:3,
b) SEQ ID NO:2 and SEQ ID NO:4,
c) SEQ ID NO:2 and SEQ ID NO:5, or
d) SEQ ID NO:2 and SEQ ID NO:6.

10. The kit as claimed in claim 8, further comprising a nucleic acid consisting of a portion of the sequence shown in SEQ ID NO: 1.

11. The kit as claimed in claim 8, further comprising a panel of at least 2 detection probes wherein at least one of the detection probes is complementary to at least a portion of a sequence in said amplification product.

12. The kit as claimed in claim 11, wherein said panel comprises at least one detection probe that is complementary to the sequence of at least one influenza A target to be amplified for each virus of said variants of Influenza A.

13. The kit as claimed in claim 11, wherein said panel comprises at least one detection probe that is substantially complementary to the sequence of at least one influenza A target to be amplified for each virus of said variants of Influenza A.

14. A method of detecting the presence of at least 90% of variants of influenza A virus in a biological sample comprising:
amplifying at least one influenza A target nucleic acid from said biological sample using primer pairs directed against a conserved region of the viral genome, wherein said region is conserved across more than at least two variants of the virus, such that at least one forward primer and at least one reverse primer in combination will bind to target genome of said variants; and
detecting any amplified target nucleic acid.

15. A method of detecting the presence of at least 90% of variants of influenza A virus in a biological sample comprising:
amplifying at least one influenza A target nucleic acid using at least one primer directed against a conserved region of the viral genome, wherein said region is conserved across more than at least two variants of the virus; and
detecting any amplified target nucleic acid.

16. The method of claim 14 or 15 wherein said variants of influenza A are selected from a group comprising: human influenza A, avian influenza A or swine influenza A.

17. The method of claim 14 or 15, wherein amplified target nucleic acid is detected by:
placing any amplified nucleic acid in contact with a panel of detection probes wherein at least one of the detection probes is complementary to at least a portion of a sequence in the amplified nucleic acid;
and
determining whether a signal indicative of the presence of said target nucleic acid in said sample has been generated.

18. The method of claim 17, wherein said panel comprises at least one detection probe that is complementary to the sequence of at least one influenza A target to be amplified, for each virus of said variants of influenza A.

19. The method of claim 17, wherein said panel comprises at least one detection probe that is substantially complementary to the sequence of at least one influenza A target to be amplified, for at least one virus of said variants of influenza A.

20. The method of claim 17, wherein at least one of said detection probes is complementary to at least a portion of a nucleic acid having the sequence or the complement of said conserved region.

21. The method of claim 17, wherein at least one of said detection probes is complementary to one or more tag sequences in the amplified DNA.

22. The method of claim 17, wherein said detection probes comprise both sequence complementary to said amplified target DNA and a tag sequence capable of hybridizing to a universal detector probe.

23. The method of claim 17, wherein at least one of said detection probes comprises at least 10 consecutive nucleotides selected from the group comprising SEQ ID NOs: 7 or 8.

24. The method of claim 17, wherein said signal is generated by a catalytic detection reagent which can produce a plurality of signals without being exhausted.

25. The method of claim 14 or 15, wherein said conserved region comprises a portion of the sequence shown in SEQ ID NO: 1.

26. The method of claim 14 wherein at least one of said primer pairs comprises:
a) SEQ ID NO:2 and SEQ ID NO:3,
b) SEQ ID NO:2 and SEQ ID NO:4,
c) SEQ ID NO:2 and SEQ ID NO:5, or
d) SEQ ID NO:2 and SEQ ID NO:6.

27. The method of claim 15 wherein said at least one primer comprises:
a) SEQ ID NO:2;
b) SEQ ID NO:3;
c) SEQ ID NO:4;
d) SEQ ID NO:5; or
e) SEQ ID NO: 6

28. A kit for detecting influenza A virus, comprising at least one primer according to claim 1, capable of participating in the production of an amplification product; and a detection reagent capable of detecting the formation of the amplification product.

29. A kit for detecting influenza A virus, comprising a set of primers according to claim 1, capable of participating in the production of an amplification product; and a detection reagent capable of detecting the formation of the amplification product.
